(19) United States
(12) Patent Application Publication
(10) Pub. No.: US 2025/0249180 A1
(43) Pub. Date: Aug. 7, 2025

(54) Cap for Removing a Needle Shield From a Medical Container, Autoinjector Including Said Cap, and Method for Assembling Said Medical Container and Said Autoinjector

(71) Applicant: Becton Dickinson France, Le Pont-de-Claix (FR)

(72) Inventor: Franck Carrel, Saint Jean de Vaulx (FR)

(21) Appl. No.: 18/855,913

(22) PCT Filed: Apr. 5, 2023
(86) PCT No.: PCT/EP2023/059047

§ 371 Date: Oct. 10, 2024

(30) Foreign Application Priority Data
Apr. 11, 2022 (EP) 22305520.3

(51) Int. Cl. A61M 5/32; A61M 5/20

(52) U.S. Cl. CPC A61M 5/3204; A61M 5/2033; A61M 2207/00

(57) ABSTRACT

The cap includes a tubular body extending along a longitudinal axis A. The tubular body has a distal end and an opposite proximal end for attachment to a bottom housing of an autoinjector. An inner sleeve proximally extends inside the tubular body from said distal end, the inner sleeve defining an inner cavity for receiving the needle shield. The cap has clamping means for clamping the needle shield when the needle shield is inserted inside the inner cavity, the clamping means including an axial abutment surface for abutting against a proximal end of the needle shield, and a plug at least partially extending inside the inner cavity, the plug having a distal end portion, a proximal end portion and an intermediate portion extending between the distal end portion and the proximal end portion. At least the proximal end portion of the plug is movable between a first position, in which the proximal end portion allows the proximal end of the needle shield to distally pass the axial abutment surface of the clamping means, and a second position, in which the proximal end portion of the plug moves in the proximal direction to push the needle shield back towards the axial abutment surface.

### CROSS-REFERENCE TO RELATED APPLICATIONS

This application is the United States national phase of International Patent Application No. PCT/EP2023/059047 filed Apr. 5, 2023, and claims priority to European Patent Application No. 22305520.3 filed Apr. 11, 2022, the disclosures of which are hereby incorporated by reference in their entireties.

### BACKGROUND OF THE INVENTION

### Field of the Invention

The present invention relates to a cap for removing a needle shield from a medical container, an autoinjector provided with this cap, and a method for assembling a medical container and the autoinjector.

### Description of Related Art

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

Automatic injection devices are designed for automatic injection of a medical product into an injection site. Autoinjectors usually comprise a housing for receiving a medical container. The medical container has a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to respectively shield or unveil the needle and an injection mechanism for automatically injecting the medical into an injection site. The injection mechanism usually includes a plunger rod for pushing a stopper inside the barrel of the medical container, and an initially compressed spring for moving the plunger in the distal direction. Locking means are provided for maintaining the plunger rod in an initial position in which the plunger rod is axially blocked despite the action of the compressed spring. A release member is typically arranged to release the plunger from the locking means and allow the spring to push the plunger rod in the distal direction to perform injection. A predetermined displacement of the safety shield towards the retracted position is required to allow the release member to unlock the locking means and release the plunger rod.

The document EP2921191 discloses a device for automatic injection of a medical product into an injection site. The device includes a two-part assembly having an upper sub-assembly assembled to a lower sub-assembly. The lower sub-assembly receives a syringe provided with an injection needle. The injection needle is covered by a rigid needle shield. The device further includes a needle cover movable relative to the housing between an extended and a retracted position. The plunger rod is coupled to a compressed spring and maintained in an initial position by flexible teeth of an inner cylinder. A push button is arranged at a proximal end of the device for releasing the plunger rod by deflecting the flexible teeth outwardly. Activation of the push button only becomes possible when the needle cover has moved a predetermined distance in the proximal direction towards the retracted position. The device also includes a cap provided with clamping means for abutting against a proximal end of the rigid needle shield such that removal of the cap entails removal of the rigid needle shield. The cap includes a central hole for accommodating the rigid needle shield during assembly.

The syringe is distally inserted into the lower sub-assembly. During this insertion, the clamping means of the cap are deflected by the syringe barrel. If nothing is done, the clamping means may tend to creep over time, i.e. stay deflected, the risk being that later, the clamping means fail to properly engage the needle shield during removal of the cap. There is therefore a need for a “push back” step in which the rigid needle shield is pushed back in the proximal direction so that the clamping means may return to a rest position in which the clamping means can reliable engage the needle shield. This push-back step may be operated by a machine engaging the central hole of the cap for pushing the needle shield back in the proximal direction. However, this operation is time-consuming. Besides, the central hole of the cap makes the needle shield visible to the end user. This may be confusing for the end user who is generally not a skilled healthcare worker.

The document GB2465389 discloses a syringe needle cover remover. The document WO2005079889 discloses a handling device for use with a medical injector including a protective sheath.

There is therefore a need for a cap that enables to avoid the above-mentioned creep effect and that permits a more efficient push-back step during assembly of the medical container within the autoinjector.

### SUMMARY OF THE INVENTION

An aspect of the invention is a cap for removing a needle shield attached to a medical container provided with an injection needle, the cap including:
a tubular body extending along a longitudinal axis A, the tubular body having a distal end and an opposite proximal end for attachment to a bottom housing of an autoinjector,
an inner sleeve proximally extending inside the tubular body from said distal end, the inner sleeve defining an inner cavity for receiving the needle shield,
clamping means for clamping the needle shield when the needle shield is inserted inside the inner cavity, the clamping means including an axial abutment surface for abutting against a proximal end of the needle shield,
a plug at least partially extending inside the inner cavity, the plug having a distal end portion, a proximal end portion and an intermediate portion extending between the distal end portion and the proximal end portion,
wherein at least the proximal end portion of the plug is movable between a first position, in which the proximal end portion allows the proximal end of the needle shield to distally pass the axial abutment surface of the clamping means, and a second position, in which the proximal end portion of the plug moves in the proximal direction to push the needle shield back towards the axial abutment surface.

The cap of the invention thus enables to push the needle shield and the medical container back in the proximal direction such that the clamping means may return to their rest position, in which the hooks extend between a distal shoulder of the syringe barrel and the proximal end of the needle shield. As a result, the clamping means do not remain in a deflected state over a long time. Instead, the clamping means stay in their rest position, i.e. undeformed, during the storage period. When the end user pulls the cap in the distal direction, the hooks can thus reliably engage the proximal end of the needle shield. This engagement entails removal of the needle shield.

The cap of the invention may further include some or all of the features below.

In an embodiment, the distal end portion of the plug and a distal opening of the inner cavity have a complementary shape.

In an embodiment, the intermediate portion of the plug includes one or more axial walls for centering and securing the plug inside the inner cavity.

In an embodiment, the proximal end portion of the plug includes a chamfer.

In an embodiment, the distal end portion of the plug is flush with regard to the distal end of the tubular body in the second position.

In an embodiment, the distal end portion of the plug has a proximal shoulder for abutting against an axial shoulder of the inner cavity in the second position.

In an embodiment, a diameter of the proximal end portion is lower than a diameter of the distal end portion.

In an embodiment, the proximal end portion and the distal end portion of the plug are symmetrical with regard to a transversal plane orthogonal to the longitudinal axis A.

The proximal end portion and the distal end portion of the plug may thus be identical.

In an embodiment, the intermediate portion of the plug includes a spring portion for biasing the proximal end portion in the proximal direction.

In an embodiment, the proximal end portion of the plug is disc-shaped.

In an embodiment, the proximal end portion of the plug is formed by a proximal end of said one or more axial walls.

In an embodiment, the plug is made of a single piece.

In an embodiment, the plug is rotationally symmetrical around the longitudinal axis A.

The plug may be axially movable between a pre-assembled position in which the proximal end portion is away from the needle shield and an assembled position in which the proximal end portion abuts against the needle shield. In the pre-assembled position, the plug may partially extend outside the inner cavity and may protrude from the distal end of the tubular body, while in the assembled position, the plug may be fully inserted inside the inner cavity.

Another aspect of the invention is an autoinjector including a lower sub-assembly provided with the above-described cap.

Another aspect of the invention is a method for assembling the above-described autoinjector and a medical container having a needle shield, the method including the steps of:
inserting the medical container inside the lower sub-assembly of the autoinjector and the needle shield inside the inner cavity of the cap until a proximal end of the needle shield distally passes over the axial abutment surface of the clamping means;
pushing the needle shield back in the proximal direction towards the axial abutment surface of the clamping means, this movement of the needle shield being caused by the proximal end portion of the plug moving from the first position to the second position.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1A is a perspective cross-section view of an autoinjector lower sub-assembly including a cap according to an embodiment of the invention,

FIG. 1B is a perspective view of a plug for a cap according to an embodiment of the invention,

FIGS. 1C and 1D are cross-section views of a cap according to an embodiment of the invention, respectively in pre-assembled position and an assembled position,

FIG. 2A is a perspective view of a plug for a cap according to an embodiment of the invention,

FIG. 2B is a cross-section view of a cap according to an embodiment of the invention, in an assembled position,

FIG. 3A is a perspective view of a plug for a cap according to an embodiment of the invention,

FIGS. 3B and 3C are cross-section views of a cap according to an embodiment of the invention, respectively in a first position and in a second position.

### DESCRIPTION OF THE INVENTION

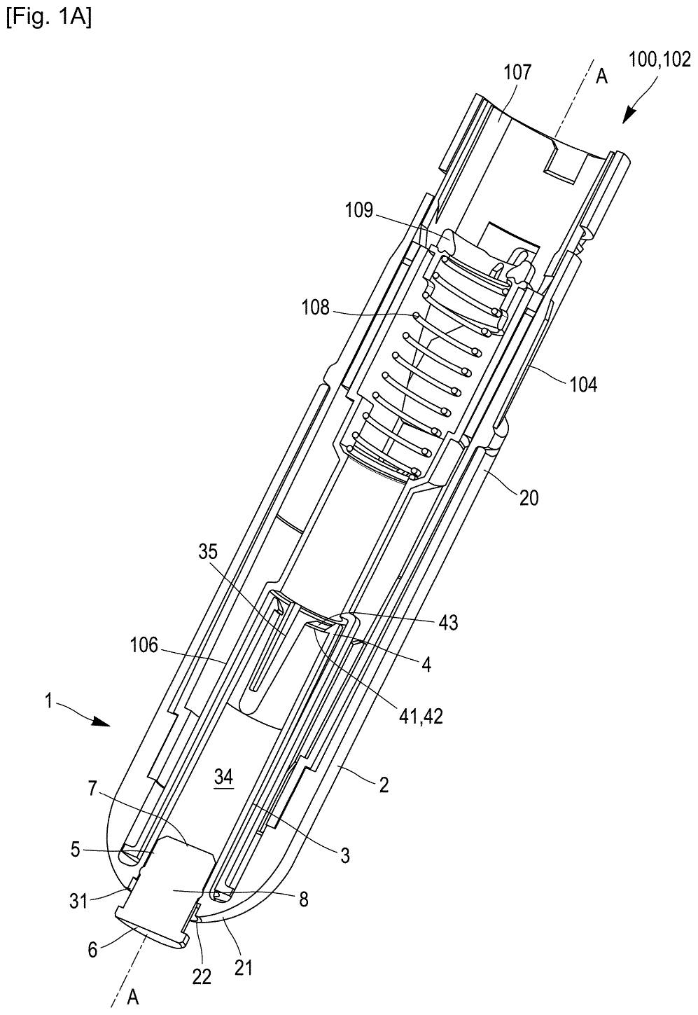

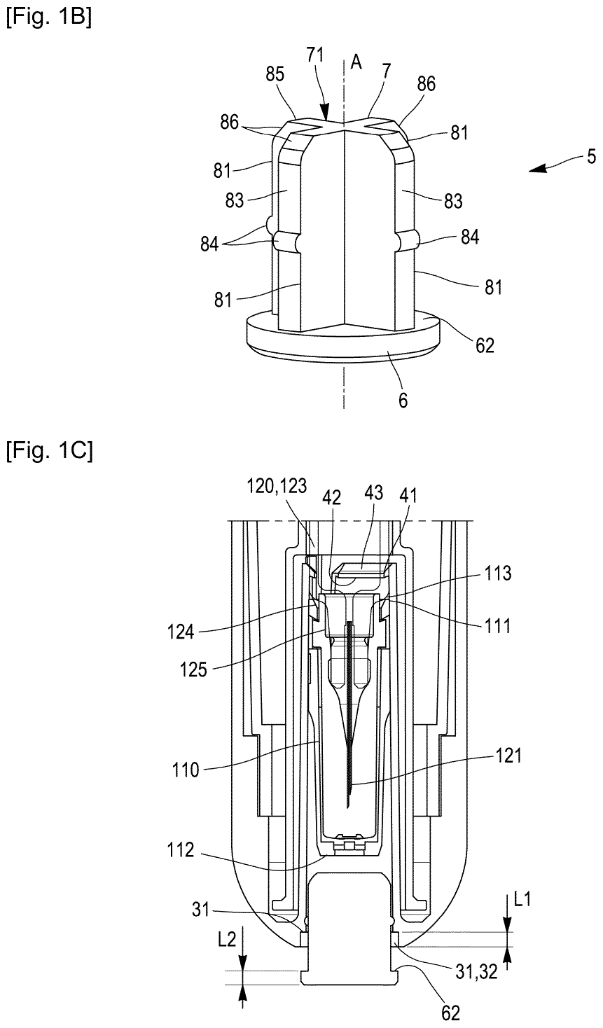

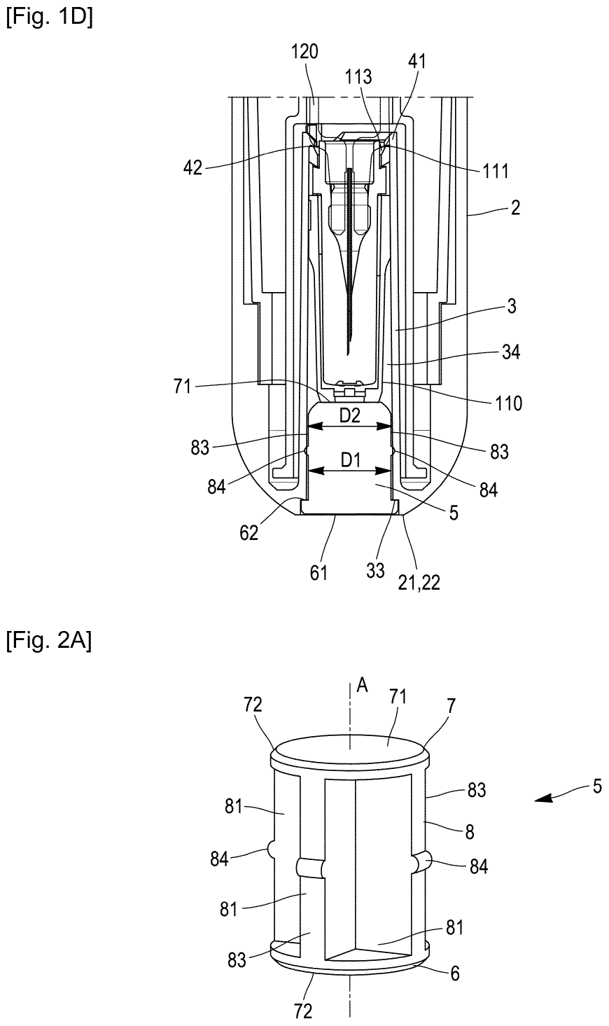

With reference to FIG. 1A-1D is shown a cap 1, according to an embodiment of the invention, for removing a needle shield 110 from a medical container 120 having an injection needle 121.

As visible in FIG. 1A, the cap 1 is intended to equip a lower sub-assembly 102 of an autoinjector 100 extending along a longitudinal axis A. The lower sub-assembly 102 of the autoinjector 100 is configured to receive a medical container 120 (FIGS. 1C and 1D), such as a prefilled syringe. As visible in FIG. 1A, the lower sub-assembly 102 may include a bottom housing 104, a needle cover 106, axially movable relative to the lower sub-assembly 102 between an extended position, in which the needle cover 106 is intended to cover the injection needle 121, and a retracted position, in which the needle cover 106 unveils the injection needle 121, a safety spring 108 for moving the needle cover 106 in the distal direction, a ring 109 intended to receive a flange of the medical container 120 so as to move axially together with the medical container 120, and a cam 107 rotationally movable around the longitudinal axis A, movement of the cam 107 being caused by the needle cover 106 moving between the extended and retracted positions.

With reference to FIGS. 1C and 1D, The medical container 120 has a barrel 123 forming a reservoir for containing a medical product and a longitudinal tip distally protruding from a distal shoulder 124 of the barrel 123. The distal tip 125 defines an axial passageway in fluid communication with the reservoir. The medical container 120 further includes an injection needle 121 inserted in the axial passageway. A needle shield 110 is removably attached to the distal tip 125 of the medical container 120 to seal and protect the needle.

The needle shield 110 has a distal end 112 and an opposite proximal end 111. The proximal end 111 of the needle shield 110 and the distal shoulder 124 of the barrel 123 are configured to face each other and are separated by an axial gap 113.

As visible in FIG. 1A, the cap 1 includes a tubular body 2 having a distal end 21 and an opposite proximal end 20 for attachement to the bottom housing 104 of the autoinjector 100. The cap 1 may be attached to the bottom housing 104 by any appropriate means such, for example, friction-fit or snap-fitting means. The distal end 21 may include a circumferential flat surface 22, orthogonal to the longitudinal axis A, which may delimit a distal opening 31.

The cap 1 also includes an inner sleeve 3 axially extending inside the tubular body 2, from the distal end 21 of the cap 1. The tubular body 2 and the inner sleeve 3 may be made of a single piece. The inner sleeve 3 defines an inner cavity 34 configured to receive the needle shield 110. The inner cavity 34 leads to an opened proximal end 35 for allowing the needle shield 110 to penetrate inside the inner sleeve 3. Opposite said opened proximal end 35, the inner cavity 34 leads to the distal opening 31. The inner sleeve 3 has clamping means, such as one or more axially extending fexible legs 4, configured to clamp the needle shield 110. The clamping means are configured such that removal of the cap 1 from the bottom housing 104 entails removal of the needle shield 110 from the medical container 120. The flexible legs 4 may be regularly distributed around the longitudinal axis A. There may be four legs 4 as shown in FIG. 1A, although the number of legs 4 may vary and may be less than four or more than four.

The flexible legs 4 have a hook 41 arranged at their proximal end. The flexible legs 4 are radially movable between a rest position, in which the hooks 41 are configured to engage the axial gap 113 separating the barrel 123 and the needle shield 110, and an outwardly deflected position, in which the legs 4 are deflected to allow insertion of the needle shield 110 inside the inner cavity 34. The hooks 41 include an axial abutment surface 42 configured to face and/or abuts against the proximal end 111 of the needle shield 110 and, opposite the axial abutment surface 42, a proximal ramp 43 inclined with respect to the longitudinal axis A for allowing the legs 4 to outwardly deflect when the needle shield 110 is introduced inside the inner sleeve 3.

With reference to FIGS. 1A and 1B, the cap 1 includes a plug 5 arranged inside the inner cavity 34 for closing the distal opening 31. As a result, the plug 5 hides the needle shield 110 to the end user. With reference to FIGS. 1B, 2A or 3A, the plug 5 includes a distal end portion 6, an opposite proximal end portion 7 and an intermediate portion 8 axially extending between the distal and the proximal end portions 6, 7.

The plug 5 of FIG. 1B is axially movable with regard to the inner sleeve 3 between a pre-assembled position (FIG. 1C) in which the plug 5 partially extends outside the inner cavity 34 and distally protrudes from the distal end 21 of the cap 1, and an assembled position (FIG. 1D) in which the plug 5 is fully inserted within the inner sleeve 3.

As illustrated in FIG. 1B, the distal end portion 6 of the plug 5 is shaped to seal the distal opening 31. To that end, the distal end portion 6 of the plug 5 and the distal opening 31 may be complementarily-shaped, and for instance the distal end portion 6 of the plug 5 may be disc-shaped to fit the distal opening 31. The distal end portion 6 of the plug 5 has a distal surface configured to allow application of a proximal force for pushing the plug 5 in the proximal direction inside the inner cavity 34. This distal surface may be flat and orthogonal to the longitudinal axis A.

With reference to FIG. 1C, the distal opening 31 of the cap 1 may include an axial shoulder 33 for stopping insertion of the plug 5 inside the inner cavity 34 at a predetermined location. The axial shoulder 33 may delimit a recess 32 for accommodating the distal end portion 6 of the plug 5, which may be complementarily shaped with said recess 32. More specifically, the axial dimension L1 of the recess 32 may be similar to the axial dimension L2 of the distal end portion 6. The plug 5 thus does not distally protrude from the distal end 21 of the cap 1 when in the assembled position (FIG. 1D), i.e. when a proximal shoulder 62 of the distal end portion 6 abuts against the axial shoulder 33 of the distal opening 31. Preferably, the distal suface 61 of the plug 5 is flush with the distal end 21 of the body (and more specifically with the circumferential flat surface 22 of said distal end 21).

The intermediate portion 8 of the plug 5 is configured to center and maintain the plug 5 inside the inner cavity 34. As illustrated in FIG. 1B, the intermediate portion 8 of the plug 5 may include one or more axial walls 81 extending parallel to the longitudinal axis A. In the embodiment shown in FIG. 1B, the plug 5 comprises four axial walls 81, however the plug 5 may include more or less than four axial walls 81, such as for instance three, five or six axial walls 81. The axial walls 81 may include the central longitudinal axis A and cross each other along said central longitudinal axis A. The axial walls 81 may extend at regular angles from each other around the longitudinal axis A, for instance they may be separated by a 90° angle as shown in FIG. 1B.

The plug 5, more specifically the intermediate portion 8 of the plug 5, further includes securing means for securing the plug 5 inside the inner cavity 34. The securing means may include an axial edge 83 of the axial walls 81. Furthermore, they may include one or more outwardly extending radial protrusions 84 configured to abut against a lateral surface of the inner cavity 34 or to engage a recess 32 (not shown) arranged on said lateral surface. To that end, the diameter D1 (see FIG. 1D) between two diametrically opposite axial edge 83 or two diametrically opposite protrusions may be higher than an inner diameter D2 of the inner sleeve 3. Preferably, the securing means are thus friction-fit or snap-fitting means. As illustrated in FIG. 1B, the radial protrusions 84 may be arranged on the axial edge 83 of the axial walls 81, and for instance at the middle of the axial length of the plug 5.

The axial edges 83 of the axial walls 81 are arranged at a distance from an edge of the distal end portion 6, thereby delimiting the proximal shoulder 62 of the distal end portion 6 of the plug 5.

Still with reference to FIG. 1B, the axial walls 81 may include a chamfer 86 at their proximal end 85 for easing insertion of the plug 5 inside the inner sleeve 3.

In the embodiment illustrated in FIG. 1B, the proximal end portion 7 is defined by the proximal end 85 of the axial walls 81. The proximal end portion 7 has a proximal end surface 71 configured to act as a pushing surface for pushing the needle shield 110 in the proximal direction when the plug 5 is being moved to the assembled position (FIG. 1D). Preferably, the proximal end surface 71 is flat and orthogonal to the longitudinal axis A. Since the plug 5 includes four axial walls 81 in the embodiment illustrated in FIG. 1B, the proximal end surface 71 is X-shaped.

The pushing force exerted by the plug 5 on the needle shield 110 causes the needle shield 110, together with the medical container 120, to move back in the proximal direction until the proximal end 111 of the needle shield 110 abuts against the axial abutment surface 42 of the hooks 41 or is only separated of the axial abutment surface 42 of the hooks 41 by manufacturing tolerances. As a result, the hooks 41 engage the axial gap 113 between the barrel 123 and the needle shield 110 and the legs 4 return to their rest position. The risks are therefore avoided that a creep effect occurs to the flexible legs 4 over time. Besides, the push-back force is exerted directly on the plug 5, instead of being directly exerted on the needle shield 110.

To that end, the proximal end portion 7 of the plug 5 is axially movable between a first position (FIG. 1C) in which the proximal end portion 7 allows the proximal end 111 of the needle shield 110 to go past the the axial abutment surface 42 of the clamping means in the distal direction, and a second position (FIG. 1D), proximal to the first position, in which the proximal end portion 7 of the plug 5 abuts against the needle shield 110 and moves the needle shield 110 back in the proximal direction towards or against the axial abutement surface of the clamping means. Here, since all the plug 5 is movable, the first position corresponds to the pre-assembled position of the plug 5 and the second position corresponds to the assembled position of the plug 5. In the second position, the proximal end portion 7 is closer to the axial abutment surface 42 than in the first position.

The operation of the cap 1 is shown in FIGS. 1A, 1C and 1D.

In FIG. 1A, the bottom housing 104 has not yet received the medical container 120. The plug 5 is temporarily maintained in the pre-assembled position by the securing means, i.e. the radial protrusions 84, which engage the lateral wall of the inner cavity 34 at a first predetermined location. The proximal shoulder 62 of the plug 5 is away from the axial shoulder 33 of the distal opening 31.

In FIG. 1C, the medical container 120 has been introduced in the lower sub-assembly 102 and pushed in the distal direction until the ring 109 abuts against the rotating cam 107. The needle shield 110 has passed over the hooks 41 of the clamping means, and the legs 4 have accordingly moved to their deflected position. The hooks 41 now rest against the barrel 123 of the medical container 120 and therefore cannot return to their rest position. The plug 5 has not moved yet and is still in the pre-assembled position. The proximal end surface 71 of the plug 5 may also be away from the needle shield 110.

In FIG. 1D, a proximal pushing force has been exerted on the distal end surface 61 of the plug 5. As a result, the plug 5 has moved in the proximal direction. The proximal end surface 71 of the plug 5 has come into contact with the needle shield 110 and pushed the needle shield 110 and the medical container 120 back in the proximal direction until the plug 5 is stopped by the axial shoulder 33 of the distal opening 31. As a result of the proximal movement of the needle shield 110 and the medical container 120 due to the pressure of the plug 5, the axial gap 113 between the barrel 123 and the needle shield 110 has moved at the level of the hooks 41, which now engage this axial gap 113. The legs 4 are no longer deflected and have returned to their rest position. The axial abutment surface 42 of the hooks 41 now faces the proximal end 111 of the needle shield 110. Thus, withdrawal of the cap 1 will reliably entail removal of the needle shield 110.

As visible in FIG. 1D, the plug 5 is now in the assembled position. The proximal shoulder 62 of the plug 5 abuts against the axial shoulder 33 of the cap 1. The proximal end surface 71 of the plug 5 abuts against the needle shield 110. The plug 5 is maintained in the assembled position by the securing means which engage the lateral wall of the inner cavity 34 at a second predetermined location proximally located relative to the first predetermined location. Due to the abutment between the plug 5 and the needle shield 110, the needle shield 110 and the medical container 120 cannot move in the distal direction during the storage period. The legs 4 remain in their rest position and risks that a creep effect occurs are avoided.

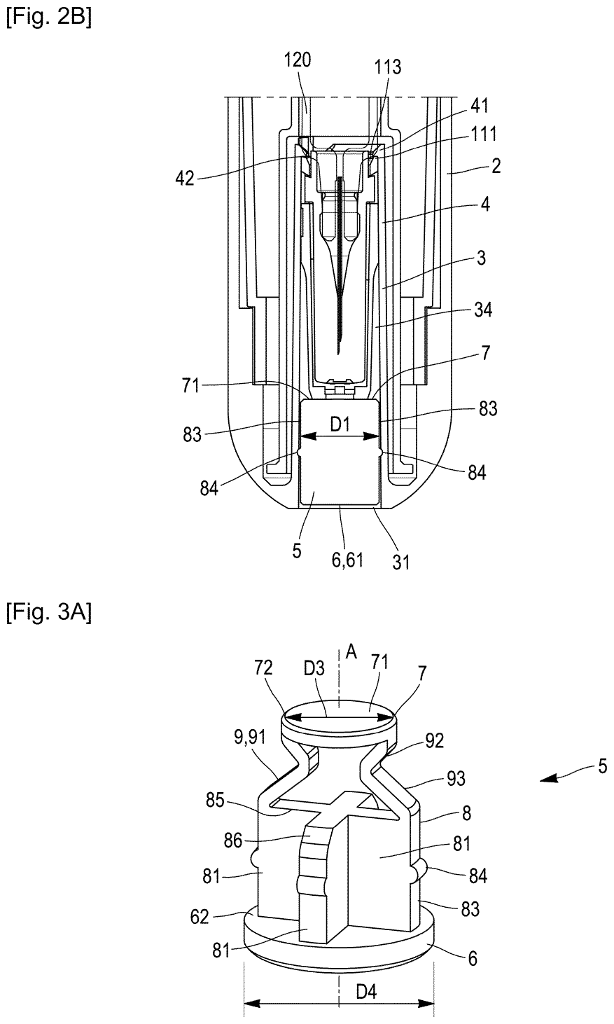

Turning now to FIGS. 2A and 2B is shown a plug 5 according to another embodiment of the cap 1 of the invention. Similar features with the other embodiments are designated by the same numeral references.

Just as the embodiment of FIGS. 1A to 1D, the plug 5 of FIG. 2A is axially movable with regard to the inner sleeve 3 between a pre-assembled position (not shown) in which the plug 5 partially extends outside the inner cavity 34 and distally protrudes from the distal end 21 of the cap 1, and an assembled position (FIG. 2B) in which the plug 5 is fully inserted within the inner sleeve 3.

The plug 5 illustrated in FIG. 2A is similar to the plug 5 shown in FIG. 1A, except that the proximal end portion 7 is also shaped to fit the inner cavity 34 and seal the distal opening 31. That is, the proximal end portion 7 and the distal end portion 6 of the plug 5 are similar. The plug 5 is symmetrical with regard to a transversal plane orthogonal to the longitudinal axis A. As a result, it is indifferent whether the distal end portion 6 or the proximal end portion 7 of the plug 5 is inserted first. There is no need to turn the plug 5 upside down before assembly. This simplifies the assembly of the cap 1.

More specifically, the distal end portion 6 has no proximal shoulder 62. The diameter of the proximal end portion 7 is the same as the diameter D1 defined by two diametrically opposite axial edge 83 of the axial walls 81. It is also the same as the diameter of the distal end portion 6. The axial edge 83 of the axial walls 81 and the edges of the distal end portion 6 and the proximal end portion 7 accordingly extend at the same distance from the central longitudinal axis A.

Besides, as visible in FIG. 2B, the distal opening 31 of the cap 1 has no recess 32 for accommodating the distal end portion 6 of the plug 5 and thus the inner cavity 34 is devoid of an axial shoulder 33. As a result, insertion of the plug 5 inside the inner cavity 34 is stopped by the proximal end 111 of the needle shield 110 abutting against the axial abutment surface 42 of the hooks 41.

It is also contemplated that the proximal end portion 7, and the similar distal end portion 6, has a chamfer 72 extending around the proximal end surface 71, respectively around the. This chamfer 72 eases insertion of the plug 5 inside the inner cavity 34.

In the pre-assembled position, the plug 5 is temporarily maintained by the securing means, i.e. the radial protrusions 84, at a first predetermined location. The proximal end surface 71 of the plug 5 may be away from the needle shield 110.

In the assembled position (FIG. 2B), a proximal pushing force has been exerted on the distal end surface 61 of the plug 5. As a result, the plug 5 has moved in the proximal direction. The proximal end surface 71 of the plug 5 has come into contact with the needle shield 110 and pushed the needle shield 110 and the medical container 120 back in the proximal direction until the plug 5 is stopped by the axial abutment surface 42 of the hooks 41 which abuts against the proximal end 111 of the needle shield 110. As a result of the proximal movement of the needle shield 110 and the medical container 120 due to the pressure of the plug 5, the axial gap 113 between the barrel 123 and the needle shield 110 has moved at the level of the hooks 41, which now engage this axial gap 113. The legs 4 are no longer deflected and have returned to their rest position. Thus, withdrawal of the cap 1 will reliably entail removal of the needle shield 110.

To that end, the proximal end portion 7 of the plug 5 is axially movable between a first position (not shown) in which the proximal end portion 7 allows the proximal end of the needle shield 110 to go past the the axial abutment surface 42 of the clamping means in the distal direction, and a second position (FIG. 2B), proximal to the first position, in which the proximal end portion 7 of the plug 5 abuts against the needle shield 110 and moves the needle shield 110 back in the proximal direction towards or against the axial abutement surface of the clamping means. Here, since all the plug 5 is movable, the first position corresponds to the pre-assembled position of the plug 5 and the second position corresponds to the assembled position of the plug 5. In the second position, the proximal end portion 7 is closer to the axial abutment surface 42 than in the first position.

As visible in FIG. 2B, the proximal end surface 71 of the plug 5 abuts against the needle shield 110. The plug 5 is maintained in the assembled position by the securing means which engage the lateral wall of the inner cavity 34 at a second predetermined location proximally located relative to the first predetermined location. Due to the abutment between the plug 5 and the needle shield 110, the needle shield 110 and the medical container 120 cannot move in the distal direction during the storage period. The legs 4 remain in their rest position and risks that a creep effect occurs are avoided.

Unlike the embodiment of FIG. 1D, the distal end surface 61 of the plug 5 in the assembled position may however not be flush with the distal end 21 of the cap 1. Instead, this distal end surface 61 of the plug 5 may be located slightly proximal to said distal end 21 of the cap 1.

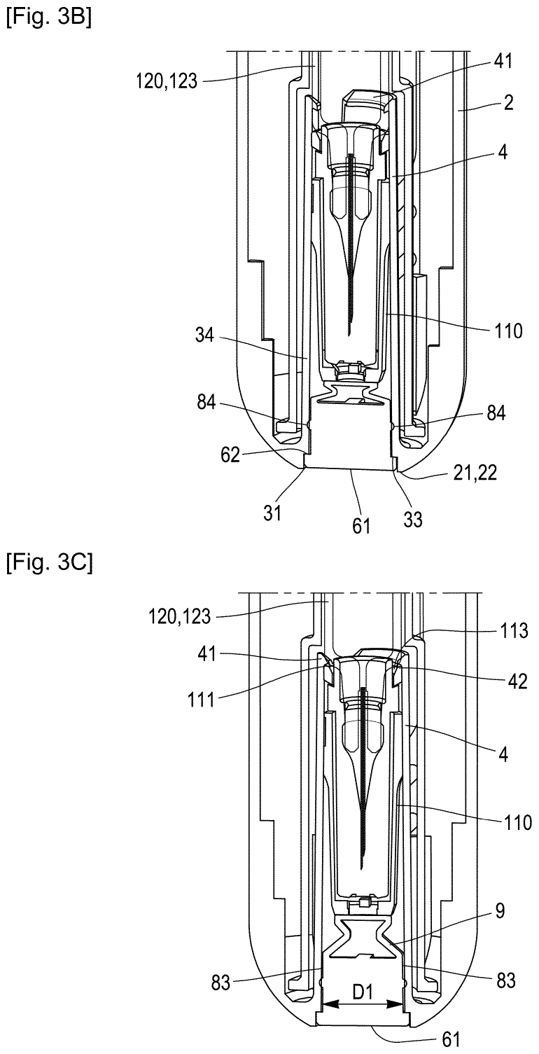

Turning now to FIGS. 3A-3C is shown a plug 5 according to another embodiment of the cap 1 of the invention. Similar features with the other embodiments are designated by the same numeral references.

The plug 5 of FIGS. 3A-3C is similar to the plug 5 shown in FIGS. 1A-1D, except that the plug 5 of FIGS. 3A-3C further includes a spring portion 9. The spring portion 9 is configured to resiliently bias the proximal end portion 7 of the plug 5 in the proximal direction. The spring portion 9 may be arranged between the intermediate portion 8 and the proximal end portion 7 of the plug 5.

With reference to FIG. 3A, the spring portion 9 includes two resiliently deformable arms 91, movable between a rest position (FIG. 3A), in which the spring portion 9 maintains the proximal end portion 7 at a stable axial position, and a deformed position (FIG. 3B), in which the spring portion 9 is compressed and in which the proximal end portion 7 of the plug 5 has moved distally relative to the stable axial position. The spring portion 9 may be V-shaped. The arms 91 may include an upper and a lower inclined branches joining each other towards the central longitudinal axis A. The upper branch 92 may be shorter than the lower branch 93. The upper branch 92 connects the lower branch 93 to the proximal end portion 7 (and more specifically to a distal surface of the proximal end portion 7), while the lower branch 93 connects the upper branch 92 to the intermediate portion 8 (and more specifically to a proximal end 85 of the axial walls 81).

As visible in FIG. 3A, the proximal end portion 7 of the plug 5 is not formed by the proximal end surface 71 of the axial walls 81, but is instead separated from the axial walls 81 by the spring portion 9. This proximal end portion 7 may be disc-shaped to better distribute the effort exerted by the spring against the needle shield 110. The proximal end portion 7 preferably has a diameter D3 which is lower than a diameter D4 of the distal end portion 6 and the inner diameter D2 of the inner sleeve 3. The diameter D3 may also be lower than the diameter D1 of the axial edges 83. Thus, the proximal end portion 7 of the plug 5 does not hamper insertion of the plug 5 inside the inner cavity 34. The proximal end portion 7 may also include a chamfer 72 surrounding the proximal end surface 71 in order to ease insertion of the plug 5 inside the inner cavity 34.

The proximal end portion 7 of the plug 5 is axially movable between a first position (FIG. 3B) in which the proximal end portion 7 allows the proximal end 111 of the needle shield 110 to go past the the axial abutment surface 42 of the clamping means in the distal direction, and a second position (FIG. 3C), proximal to the first position, in which the proximal end portion 7 of the plug 5 abuts against the needle shield 110 and moves the needle shield 110 back in the proximal direction towards or against the axial abutement surface of the clamping means. Here, since the plug 5 may be fixed, the first position corresponds to the deformed position of the spring portion 9 of the plug 5 and the second position corresponds to the rest position or a less deformed position of the spring portion 9 of the plug 5. In the second position, the proximal end portion 7 is closer to the axial abutment surface 42 than in the first position.

The operation of the cap 1 is shown in FIGS. 3B and 3C.

Before insertion of the medical container 120 into the bottom housing 104, it is contemplated that the plug 5 may either be in a pre-assembled position (similar to the one illustrated in FIG. 1C) or may already be in an assembled position in which the plug 5 is fully inserted inserted inside the cap 1.

In FIG. 3B, the medical container 120 has been introduced in the bottom housing 104 and pushed in the distal direction until the ring 109 abuts against the rotating cam 107. The needle shield 110 has passed over the hooks 41 of the clamping means, and the legs 4 have accordingly moved to their deflected position. The hooks 41 now rest against the barrel 123 of the medical container 120 and therefore cannot return to their rest position.

Still with reference to FIG. 3B, the plug 5 is illustrated in the assembled position. The proximal shoulder 62 of the plug 5 abuts against the axial shoulder 33 of the distal opening 31. The plug 5 does not distally protrude from the distal end 21 of the cap 1. The distal surface of the plug 5 is flush with the circumferential flat surface 22 of the cap 1. The securing means, i.e. the radial protrusions 84, engage the inner lateral wall of the inner cavity 34 and thus maintain the plug 5 in the assembled position. Besides, the proximal end portion 7 of the plug 5 abuts against the needle shield 110 and the spring portion 9 is in the deformed position. As a result, the spring portion 9 the plug 5 exerts a proximal pushing force on the needle shield 110.

In FIG. 3C, due to the pressure of the spring portion 9, the needle shield 110 and the medical container 120 have moved back in the proximal direction until the proximal end 111 of the needle shield 110 abuts against the hooks 41. As a result of the proximal movement of the needle shield 110 and the medical container 120 due to the pressure of the spring portion 9, the axial gap 113 between the barrel 123 and the needle shield 110 has moved at the level of the hooks 41, which now engage this axial gap 113. The legs 4 are no longer deflected and have returned to their rest position. The axial abutment surface 42 of the hooks 41 now abuts against the proximal end 111 of the needle shield 110. Thus, withdrawal of the cap 1 will reliably entail removal of the needle shield 110.

In FIG. 3C, due to the abutment between the plug 5 and the needle shield 110 and the effort of the spring portion 9, the needle shield 110 and the medical container 120 cannot move in the distal direction during the storage period. The legs 4 remain in their rest position and risks that a creep effect occurs are avoided.

The embodiment of FIGS. 3A-3C allows that the push-back force is exerted by the plug 5 itself, instead of being exerted by an external machine or operator, so that there is no need for an additional push-back step during the assembly process. That is, the push-back step is automatically realized by the plug 5 itself.

As illustrated in FIGS. 1A, 2B and 3B, the cap 1 of the invention is preferably a two-part cap 1. That is, the plug 5 and the tubular body 2 of the cap 1 are preferably two distinct components. With reference to FIGS. 1B, 2A and 3A, the plug 5 may be made of a single piece, for example by injection molding. The material of the plug 5 may include acrylonitrile-butadiene-styrene (ABS), polycarbonate (PC), polyoxymethylene (POM) and any combinations thereof. The plug 5 may also include any other appropriate material. In an alternative embodiment not shown, the plug 5 of FIGS. 3A-3C might however be integral with the inner sleeve 3 and the tubular body 2

Preferably, the plug 5 is rotationally symmetrical around the longitudinal axis A (for instance a 90° or 180° rotation in the embodiments illustrated in FIGS. 1B, 2A and 3A). Accordingly, insertion of the plug 5 into the inner cavity 34 is easily done whatever the angular orientation of the plug 5 with regard to the longitudinal axis A and the inner sleeve 3.

The invention also relates to a method for assembling a medical container 120 having a needle shield 110 with an autoinjector 100 including the above-described cap 1. The method includes a step of inserting the medical container 120 inside the bottom housing 104. During this step, the needle shield 110 deflects the flexible legs 4 and enters the inner sleeve 3. Insertion of the needle shield 110 inside the inner cavity 34 is stopped by the ring 109 abutting against the cam 107 of the lower sub-assembly 102. At this stage, the needle shield 110 has passed over the hooks 41 and the proximal end 111 of the needle shield 110 is distally distant from the axial abutment surface 42 of the hooks 41. Since the hooks 41 bear against the barrel 123 of the medical container 120, and thus are in a deflected position, it is now required to push the needle shield 110 and the medical container 120 back in the proximal direction. Thus, the method includes a step of pushing the needle shield 110 back in the proximal direction by means of the plug 5. Either the plug 5 automatically pushes the needle shield 110 back by means of the spring portion 9 (FIGS. 3A-3C), or a machine or an operator moves the plug 5 from the pre-assembled to the assembled position (FIGS. 1C-1D and 2B). Movement of the plug 5 from the pre-assembled to the assembled position causes the plug 5 to abut against the needle shield 110 and push the needle shield 110 in the distal direction. Either the movement of the plug 5 is stopped by its proximal shoulder 62 abutting against the axial shoulder 33 of the cap 1 (FIG. 1D), i.e. the assembly is “stroke driven”, or the movement of the plug 5 is stopped by the needle shield 110 abutting against the hooks 41 (FIG. 2B), i.e. the assembly is “force driven”. In the first case (FIG. 1D), a small distance may still separate the hooks 41 and the needle shield 110, due to the manufacturing tolerances. In the second case (FIG. 2B), the needle shield 110 and the hooks 41 abut against each other. In all cases however, the hooks 41 have returned to their rest position and engage the room between the barrel 123 and the needle shield 110, so that the axial abutment surface 42 of the hooks 41 necessarily engage the proximal end 111 of the needle shield 110 when the cap 1 is withdrawn. This ensures removal of the needle shield 110.

It is to be understood that the present invention is not limited to the embodiments described above and illustrated in the drawings; rather, the skilled person will recognize that many changes and modifications may be made within the scope of the appended claims.

1. A cap for removing a needle shield attached to a medical container provided with an injection needle, the cap comprising:
a tubular body extending along a longitudinal axis A, the tubular body having a distal end and an opposite proximal end for attachment to a bottom housing of an autoinjector,
an inner sleeve proximally extending inside the tubular body from said distal end, the inner sleeve defining an inner cavity for receiving the needle shield,
the inner sleeve having clamping means for clamping the needle shield when the needle shield is inserted inside the inner cavity, the clamping means comprising an axial abutment surface for abutting against a proximal end of the needle shield,
a plug at least partially extending inside the inner cavity, the plug having a distal end portion, a proximal end portion and an intermediate portion extending between the distal end portion and the proximal end portion,
wherein at least the proximal end portion of the plug is movable between a first position, in which the proximal end portion allows the proximal end of the needle shield to distally pass the axial abutment surface of the clamping means, and a second position, proximal to the first position, in which the proximal end portion of the plug moves in the proximal direction to push the needle shield back towards the axial abutment surface.

2. The cap according to claim 1, wherein the distal end portion of the plug and a distal opening of the inner cavity have a complementary shape.

3. The cap according to claim 1, wherein the intermediate portion of the plug comprises one or more axial walls for centering and securing the plug inside the inner cavity.

4. The cap according to claim 1, wherein the proximal end portion of the plug comprises a chamfer.

5. The cap according to claim 1, wherein the distal end portion of the plug is flush with regard to the distal end of the tubular body in the second position.

6. The cap according to claim 1, wherein the distal end portion of the plug has a proximal shoulder for abutting against an axial shoulder of the inner cavity in the second position.

7. The cap according to claim 1, wherein a diameter of the proximal end portion is lower than a diameter of the distal end portion.

8. The cap according to claim 1, wherein the proximal end portion and the distal end portion of the plug are symmetrical with regard to a transversal plane orthogonal to the longitudinal axis A.

9. The cap according to claim 1, wherein the intermediate portion of the plug comprises a spring portion for biasing the proximal end portion in the proximal direction.

10. The cap according to claim 1, wherein the proximal end portion of the plug is disc-shaped.

11. The cap according to claim 3, wherein the proximal end portion of the plug is formed by a proximal end of said one or more axial walls.

12. The cap according to claim 1, wherein the plug is made of a single piece.

13. The cap according to claim 1, wherein the plug is rotationally symmetrical around the longitudinal axis A.

14. An autoinjector comprising a lower sub-assembly provided with the cap of claim 1.

15. A method for assembling the autoinjector of claim 14 and a medical container having a needle shield, the method comprising the steps of:
(i) inserting the medical container inside the lower sub-assembly of the autoinjector and the needle shield inside the inner cavity of the cap until a proximal end of the needle shield distally passes over the axial abutment surface of the clamping means;
(ii) pushing the needle shield back in the proximal direction towards the axial abutment surface of the clamping means, this movement of the needle shield being caused by the proximal end portion of the plug moving from the first position to the second position.
